(19) United States
(12) Patent Application Publication
(10) Pub. No.: US 2019/0224153 A1
(43) Pub. Date: Jul. 25, 2019

(54) L-DOPA DERIVATIVES FOR THE TREATMENT OF NEUROLOGICAL DISEASES

(71) Applicant: BERLIREM GMBH, Zossen (DE)

(72) Inventors: Johannes TACK, Berlin (DE); Christoph VOELKEL, Zossen (DE); Reinhard HOROWSKI, Zossen (DE); Matthias BRAEUTIGAM, Zossen (DE); Dirk PALLA, Zossen (DE)

(21) Appl. No.: 16/337,521

(22) PCT Filed: Sep. 29, 2017
(86) PCT No.: PCT/EP2017/001159

§ 371 Date: Mar. 28, 2019

(30) Foreign Application Priority Data
Sep. 29, 2016 (EP) 16002102.8
Jun. 1, 2017 (EP) 17000933.6

(51) Int. Cl. A61K 31/198; A61K 45/06; A61K 9/00

(52) U.S. Cl. CPC A61K 31/198; A61K 9/19; A61K 9/0019; A61K 45/06

(57) ABSTRACT

The present invention describes a new method and new combination products for the therapy of neurological and other conditions which respond to dopaminergic therapies and especially to L-DOPA (L-Dihydroxy-Phenylalanine) based on the use of L-DOPA esters, especially of L-DOPA glycerol ester and L-DOPA Cholin Ester.

This patent application claims the priority according to the Paris Convention of European Patent applications Nos. EP 16002102.8 filed Sep. 29, 2016 and EP 17000933.6 filed Jun. 1, 2017. The invention described herein relates to a new method and new products for the therapy of neurological and other conditions which respond to dopaminergic therapies and especially to L-DOPA (L-Dihydroxy-Phenylalanine). Especially in the dopamine(DA)-deficiency condition Parkinson's Disease (PD), oral therapy with L-DOPA is considered to be the therapeutic gold standard; however, under long term pulsatile use of this drug in PD results in severe motor fluctuations (wearing-OFF, ON-OFF, early morning OFF) and motor complications (especially peak-dose dyskinesias, but also diphasic dyskinesias and dystonias). Furthermore, oral L-DOPA sometimes fails to achieve any therapeutic effect at all, be it due to insufficient enteral absorption or to a blockade of its brain uptake e.g. by its main metabolite 3-O-Methyl-DOPA or to other factors. In a worst case this L-DOPA failure can result in an acute akinetic crisis which can be life-threatening.

Another great disadvantage of oral L-DOPA therapy is its short terminal half-life of about 30 mins which—mediated by its active metabolite DA of which L-DOPA is only a prodrug—eventually results in a pulsatile DA receptor stimulation which is the opposite from the continuous DA receptor stimulation achieved with the physiological tonic release of DA onto its receptors in the CNS motor system. This pulsatile receptor stimulation by the short-acting L-DOPA is believed to be the basis for the motor fluctuations and complications observed with chronic oral L-DOPA treatment which greatly reduce the quality of life of PD patients. It has been shown, e.g. by F. Stocchi and colleagues (for review see Olanow C W, Obeso J A and Stocchi F: Nature Clin Pract Neurol 2, 382-392, 2006), that even in fluctuating and dyskinetic PD patients, i.v. infusion of L-DOPA can abolish these problems with an even stronger anti-PD efficacy. Unfortunately, in view of the high dose needed and the chemical reactivity of L-DOPA, i.v. application is possible only for a few days, as subsequently all accessible veins will become obliterated by local reactions and thrombosis (Shoulson I, Glaubiger G A and Chase T N Neurology 25, 1144-1148, 1975).

There have been numerous attempts to increase the duration of the oral L-DOPA effect and the tolerability of this drug. These include adding peripheral decarboxylase inhibitors, COMT inhibitors and MAO-B inhibitors as well as to replace part of L-DOPA by the simultaneous use of DA agonists such as pramipexole, ropinirole or lisuride. Indeed, such complicated combination therapies are nowadays the rule in PD therapy, especially since other options such as slow-release L-DOPA products have failed to produce reliable effects. The same holds true for all other attempts to develop parenteral application forms of L-DOPA. Only a highly-concentrated suspension of L-DOPA in carboxymethylcellulose (Duodopa®) for intra-duodenal infusion is available. But this approach requires abdominal surgery and the dose is limited by the strong viscosity of this product.

Now, we have found that continuos application of L-DOPA esters, esterified with glycerol or related compounds including carbohydrates such as mono-, di- or oligosaccharides (hereinafter L-DOPA esters) surprisingly overcome all the problems of L-DOPA therapy as described here.

These current invention therefore relates to the use of compounds having the general Formula I

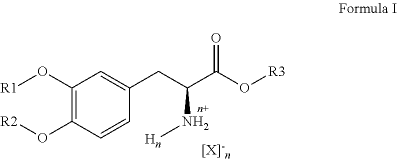

wherein [X]⁻ is a physiologically compatible anion
wherein n is 0 or 1,
wherein R1 and R2 are independently of each other, selected from the group comprising hydrogen, or hydrogensulfate, phosphate, hydrogen phosphate, dihydrogen phosphate benzoate, formate, acetate, propionate, butanoate, valerate, silyl,
or
R1, R2 together hydrogen phosphate, sulfate, methylene, isopropylidene,
wherein R3 represents a methyl-, ethyl-, n-propyl-, i-propyl-, n-butyl, i-butyl or t-butyl group or an unbranched, branched or cyclic polyhydroxyl residue with 1-12 carbon atoms and 1-6 OH-groups which can further be substituted by unsaturated groups, halogens or organic functional groups like carboxylic group and aldehyde in the treatment of neurological diseases by continuous application.

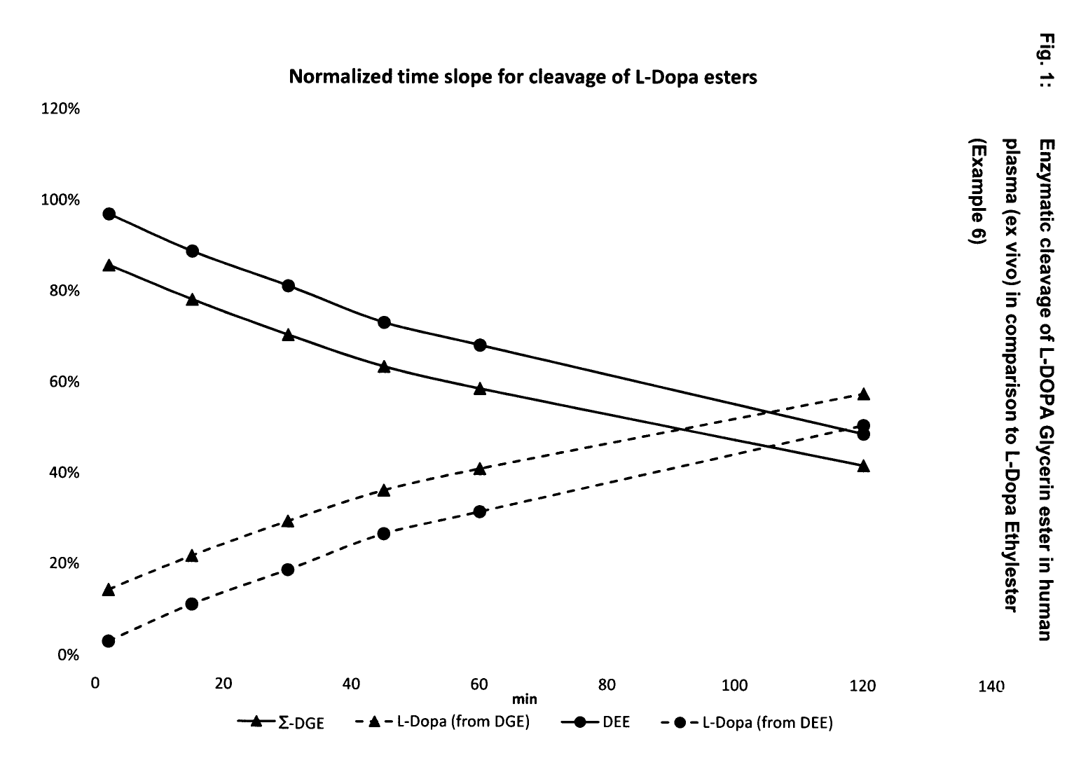

Examples of these compounds are shown in FIGS. 1a-1d of WO 2016/155888 A1, which are at the same time preferred compounds in the intended use. The compounds and various ways for their manufacture are described below as well as in EP 3075723 and WO 2016/155888A1.

The described compounds (L-DOPA esters) are highly effective in the treatment of neurological diseases such as of idiopathic PD as well as in nearly all forms of Parkinsonism (including neuroleptogenic PD where the DA antagonists can be displaced by high concentrations of L-DOPA-derived DA), Segawa's disease, Lewy Body diseases and Restless Legs Syndrome, another condition responding to dopaminergic treatments, and it is even possible to treat prolactin-producing pituitary adenomas and related disorders such as some forms of acromegaly which also can be fully controlled by stable high levels of L-DOPA.

In striking contrast to present L-DOPA products, the new L-DOPA esters can be administered in parenteral forms where they achieve long-lasting stable dopaminergic stimulation (over several days and more), when given by infusion or in pumps. The term continuous application as used herein is supposed to include all types of administration that are able to provide a substantially constant L-DOPA level for at least 48 h. Usually the methods described herein provide a substantially constant L-DOPA level for at least 96 h, preferred embodiments provide a substantially constant L-DOPA level for at least 7 days.

Another known option is to use L-DOPA ester bolus injections, preferentially s.c. but also i.m. or i.v., to achieve a well-controlled dopaminergic effect in, e.g. early morning OFF, a frequent problem in advanced PD which often is combined with dystonia and as a rule prevents the patient to start useful mobility. So far, these situations which also include short-lasting day-time OFF or even beginning akinetic crisis, were treated with apomorphine s.c. or i.m. bolus injections. However, due to the very short half-life of this drug of about 15 mins, it often proves insufficient and, as apomorphine is a potent emetic, also quite often its use is associated with nausea, emesis and orthostatism. These same adverse events also limit the alternative use of oral fast-dissolving L-DOPA (e.g. Madopar® LT) which also has a very short half-life. In contrast, the L-DOPA esters as described here can slow down the production and release of DA and thus of the associated adverse events (which are due to rapidly increasing blood levels) and also extend the intended stable efficacy to longer periods of time. Eventually it will be possible to achieve continuous dopaminergic stimulation on the basis of the new L-DOPA esters described here and thus provide PD patients with a much better symptomatic therapy and at the same time prevent long-term L-DOPA complications.

Different Esters of L-DOPA and their synthesis are mentioned since the 1970 years, claiming the principle formula and ways of synthesis (U.S. Pat. Nos. 4,035,507, 3,891,696, 5,354,885). Meanwhile it is known that for a wide range of esters described herein, the effective synthesis by these methods is not feasible. Therefore, new synthesis routes were necessary to test and develop (see examples 1-13 of WO 2016/155888A1).

Already Djaldetti et al. (Annals of Neurology Vol. 39, No. 3, 1996) used Esters of L-DOPA in pharmaceutical formulations to turn patients “on” by giving several injections subcutaneously or intramuscularly during the day.

Several efforts have been made to achieve a continuous application of L-DOPA esters, e.g. by rectal application (EP0287341A1), but with limited bioavailability. The preferential way of continuous application is by infusion or injection. In that way, continuous application systems of L-DOPA esters are already described by Heller and Heller (WO 002013/184646 A2, US 2014/0088192 A1) in form of aqueous infusible solutions, preferably the methyl and ethyl ester of L-DOPA. Disadvantage of the claimed formulations are the relatively low pH in order to get the drug stabilized in the aqueous environment.

An even easier approach is to use these compounds which have a much better solubility in water also for intraduodenal infusion instead of Duodopa® as much higher dosages can be applied which permit a 24/7 efficacy and which need much smaller, less cumbersome portable pumps. There will be no kinking of the intraduodenal tube or even full arrest of the delivery (again a risk factor for inducing an akinetic crisis) and there is also only a need for a much smaller percutaneous opening, with a significantly reduced risk of contamination and peritonitis. When used in such a way no additional toxicity studies will be necessary as the esters as prodrugs will be split already in the stomach and small intestine into L-DOPA.

A much broader spectrum of applications, however, is given by the parenteral and preferentially subcutaneous administration of these L-DOPA esters. This can be achieved by portable pumps which, as discussed, require a quite smaller volume than, e.g., in the case of s.c. apomorphine infusion or in the case of Duodopa®. They can also be injected or infused in other ways (such as disposable patch pumps (Omnipod®) or re-usable patch pumps with disposable pre-filled or fillable cartridges (Kaleido®)) into the subcutaneous tissue where they will cause much less local irritation than L-DOPA itself, and where such amounts can be applied to provide a continuous dopaminergic stimulation over many hours and even days, for the first time combining the two best options for PD therapy, i.e. the use of L-DOPA, and providing continuous dopaminergic stimulation.

Another option is to administer L-DOPA esters by an implantable mini pump (e.g. tricumed IP1000V) which is fixed in the subcutaneous tissue by a surgical intervention and delivers the active ingredient continuously or by a programmable individual profile via i.v. catheter into the arteria subclavia for example. The re-fill of the sterile solution of the active ingredient containing formulation is made through a port which is located directly under the intact skin. Sterile solutions suited for application by an implanted mini pump consists of water for injection, buffer solution or organic solvents suited for parenteral application like N-Methylpyrrolidone, Polysorbat 80, Dimethylacetamide, Solutol HS 15, Glycerol, Ethanol or mixtures with water for injection like tert.-Butanol and water 1:1. Said formulation may contain stabilizer, antioxidants and other excipients to maintain stability of the L-DOPA ester for a longer time period of for example 7 and more days.

It is an additional aspect of the invention that the high solubility of the L-DOPA ester in aprotic solvents suitable for parenteral application (such as e.g. N-Methylpyrrolidone, Polysorbat 80, Dimethylacetamide, Solutol HS 15, Glycerol, Ethanol) also relates to L-DOPA esters known before the application date of WO 2016/155888 A1, in particular L-DOPA ethyl ester described in WO 2012/079072 A2 (Heller et al.). During investigations it has been found that aprotic solutions of L-DOPA ethyl ester show a higher stability against hydrolytic cleavage compared to aqueous solutions. Moreover the stability is much higher even at pH-values of 5-8. L-DOPA ethyl ester solutions in aprotic solvents suitable for parenteral application could therefore solve tolerability problems known from earlier attempts to use L-DOPA ethyl ester in the treatment of neurological diseases.

The new administration mode of the described L-DOPA esters can be combined with peripheral decarboxylase inhibitors as well as with COMT- or MAO-B inhibitors such as entacapone, tolcapone, opicapone (preferably as only a once-a-day oral application is necessary) resp. deprenyl or rasagiline (again once-a-day). By use of non-water based formulations the said inhibitors, which have mostly limited solubility in water can be applied parenterally together with the L-DOPA ester. Another option is to apply the described inhibitors.

Surprisingly, with the blockade of the carboxyl-group by the esterification, there may be no need for a decarboxylase inhibitor at all, and also the glycerol or carbohydrate moiety will not only reduce the local concentration and local irritation by resulting in a rapid dissolution in the whole body but may also contribute to better affinities to the blood-brain barrier and their membranes (by the glycerol part) or to specific transport mechanisms as in the case of carbohydrates and thus will be more effective.

What has been discussed here in the context of an improved dopaminergic effect of the new esters of L-DOPA in PD also applies to similar esters of the noradrenaline (NA) precursor droxidopa (=L-Threo-3,4-Dihydroxy-Phenylserine) which is L-DOPA with an additional Carboxy-Group. Corresponding Di-esters can be used in a very similar way to restore the depleted NA levels in PD but also in Multisystem Atrophies (MSAs) and Orthostatic Hypotension. Such NA deficiencies have been linked to cognitive deterioration, up to dementia, depression and orthostatism, and it is obvious that also both L-DOPA and NA substitutions can be combined to achieve an even larger therapeutic spectrum in neurological and other diseases.

It may also be preferable to have the new L-DOPA Mono, Di- or oligo-esters, as have been invented earlier (EP 3075723 and WO 2016/155888 A1), with additional useful drugs for the treatment of these conditions. This applies to combinations with DA agonists and also with MAO-B inhibitors and especially with rasagiline which has been reported to exert neuroprotective properties. Rasagiline is water-soluble and, being an irreversible MAO-B inhibitor, has long-lasting effects, and therefore can be added very easily to the water-soluble esters of this invention for parenteral or other applications.

If the compounds are to be directed more exclusively to the brain, also glutathione can be used, and in such case, a strong antioxidant can be added very easily. Glutathione deficiency is one of the very first biochemical events in PD, even before significant DA depletion, and therefore its substitution will be of great therapeutic value. It also prevents the production of toxic L-DOPA autoxidation products.

As the L-DOPA esters described still undergo considerable O-Methylation, also addition of water-soluble COMT inhibitors such as entacapone, tolcapone and especially opicapone will enhance the efficacy of the new compounds. Another option is to add these L-DOPA enhancers (including selegiline and rasagiline) in their usual oral forms. This is of great importance as consumption of methyl-groups by high-dosed L-DOPA or its prodrugs will result in an accumulation of D-homo-serine which has a clear cardiovascular and probably also CNS toxicity (Mueller T et al., J Neural Transm 109, 175-179), and COMT inhibitors prevent this consumption of methyl groups.

It has furthermore been found that the described L-DOPA esters according to general formula I

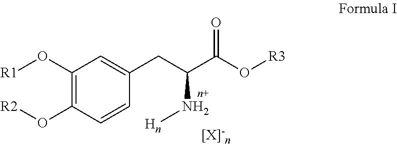

wherein [X]⁻ is a physiologically compatible anion
wherein n is 0 or 1,
wherein R1 and R2 are independently of each other, selected from the group comprising hydrogen, or hydrogensulfate, phosphate, hydrogen phosphate, dihydrogen phosphate benzoate, formate, acetate, propionate, butanoate, valerate, silyl,
or
R1, R2 together hydrogen phosphate, sulfate, methylene, isopropylidene,
wherein R3 represents a methyl-, ethyl-, n-propyl-, i-propyl-, n-butyl, i-butyl or t-butyl group or an unbranched, branched or cyclic polyhydroxyl residue with 2-12 carbon atoms and 2-6 OH-groups which can further be substituted by unsaturated groups, halogens or organic functional groups like carboxylic group and aldehyde
can be used in combination with
Decarboxylase inhibitors
   and/or
MAO-B inhibitors
   and/or
COMT inhibitors
   which could either be administered separately or could be combined within one pharmaceutical formulation as a solution or a suspension for continuous administration, e.g. intravenously, subcutaneously, sublingually, per-orally and by inhalation or any other route for example by percutaneous endoscopic gastrostomy (PEG) administration directly into the duodenum or jejunum to treat patients suffering from Parkinson's Disease (PD), Restless Legs Syndrome (RLS) or of related motor disorders or of Prolactinomas.

Preferred combinations according to such embodiment of the invention are:
The use of a compound of formula I according to claim 10, wherein the other PD therapy is s.c. apomorphine infusion or s.c. lisuride infusion
The use of a compound of formula I according to claims 2-9 wherein the MAO-B inhibitors is selegiline or rasagiline s.c. or p.o.
The use of a compound of formula I according to claims 2-9 wherein the COMT inhibitor is entacapone, colcapone, apomorphine or opicapone s.c. or p.o.

It has further been found that the described L-DOPA esters according to general formula I

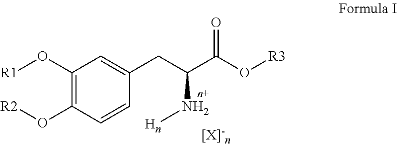

wherein [X]⁻ is a physiologically compatible anion
wherein n is 0 or 1,
wherein R1 and R2 are independently of each other, selected from the group comprising hydrogen, or hydrogensulfate, phosphate, hydrogen phosphate, dihydrogen phosphate benzoate, formate, acetate, propionate, butanoate, valerate, silyl,
or
R1, R2 together hydrogen phosphate, sulfate, methylene, isopropylidene,
wherein R3 represents a methyl-, ethyl-, n-propyl-, i-propyl-, n-butyl, i-butyl or t-butyl group or an unbranched, branched or cyclic polyhydroxyl residue with 2-12 carbon atoms and 2-6 OH-groups which can further be substituted by unsaturated groups, halogens or organic functional groups like carboxylic group and aldehyde can be used to treat patients suffering from neurological disorders, such as Restless Legs Syndrome (RLS) or of related motor disorders or of Prolactinomas. In the same way, the described compounds can be used in the prevention or treatment of OFF phenomena and especially akinetic crisis in PD patients.

Preferred embodiments of the invention are provided below and in the claims.

In conclusion, combinations provide much easier and faster dose finding and stable and clearly enhanced long-term efficacy, not affected by concomitant therapies and, surprisingly, at the same time better tolerability.

### Definition: Decarboxylase Inhibitor

An aromatic L-amino acid decarboxylase inhibitor (synonyms: DOPA decarboxylase inhibitor, DDCI and AAADI) is a drug which inhibits the synthesis of dopamine by the enzyme aromatic L-amino acid decarboxylase (AAAD, or DOPA decarboxylase, DDC).

Examples of Decarboxylase inhibitors are:
1. Benserazide (Madopar®, Prolopa®, Modopar®, Madopark®, Neodopasol®, EC-Doparyl®, etc.)
2. Carbidopa (Lodosyn®, Sinemet®, Parcopa®, Atamet®, Stalevo®, etc.) and Carbidopaethylester (EthylCarbidopa)
3. Methyldopa (Aldomet®, Aldoril®, Dopamet®, Dopegyt®, etc.)
4. α-Difluoromethyl-DOPA (DFMD, DFM-DOPA)

### Definition: MAO-B Inhibitors

Monoamine oxidase inhibitors (MAOIs) are chemicals that inhibit the activity of the monoamine oxidase B enzyme family.

Examples of MAO-B inhibitors are:
1. Rasagiline (Azilect®)
2. Selegiline (Deprenyl®, Eldepryl®, Emsam®, Zelapar®)

### Definition: COMT Inhibitor

A COMT inhibitor is a drug that inhibits the action of catechol-O-methyl transferase.

Examples of COMT inhibitors are:
1. entacapone
2. colcapone
3. opicapone
4. tolcapone
5. apomorphine
6. nitecapone.

Pharmaceutical preparations are preferably offered as sterile solutions or lyophilizates, parenteral, per-oral, microcrystalline and nanocrystalline formulations, liposomal formulations, microcapsules, emulsions, and dispersions, and they are especially suitable for subcutaneous, intravenous, per-oral, percutaneous (PEG) or pulmonary use or application.

Lactose, starch, sorbitol, mannitol, sucrose, ethyl alcohol and water can be used, for example, as pharmacologically and chemically compatible carriers, solvents or adjuvants.

In addition, starches, modified starches, gelatins, natural sugars, natural or synthetic polymers, such as, for example, acacia gum, guar, sodium alginate, carboxymethyl cellulose or polyethylene glycol, can be included as binding agents.

Cyclodextrins, modified cyclodextrins, also benzoates, chlorides, acetates, and tartrates can be included as stabilizers, and stearates, polyethylene glycol, amino acids, such as, for example, L-Cysteine or Glutathione, can be used as adjuvants, usually in concentrations of 0.05% to 15%.

Liquid formulations include solutions, dispersions and emulsions. Liquid preparations for parenteral use are sterile and contain water or water and solubilizers, such as, for example, propylene glycol, micelle formers as Polyethylenglycol (15)-hydroxystearate Solutol® HS15 and mixed micelle formers.

Starches or modified starches, alginates, aluminates, bentonites or microcrystalline cellulose can be used at concentrations of usually between 2% and 30% according to weight.

Sugar, sugar alcohols, corn, rice or potato starches, gelatins, gum arabic, tragacanth sugar, ammonium calcium alginate, carboxymethyl cellulose, hydroxypropyl methyl cellulose, polyvinyl pyrrolidone and inorganic substances can be used as adjuvants usually at concentrations of between 1% to 30% according to weight.

Manufacture of L-DOPA Esters

The L-DOPA esters required to carry out the present invention can be synthesized by the methods explained in WO 2016/155888A1, the content of which is herewith included in its entirety by reference:

If one or more chiral centers are present in a compound of formula (I), then all forms of these isomers, including enantiomers and all possible diastereomers, should be included in the context of this invention. Compounds which contain a minimum of one chiral center may be used as a racemic mixture, in this case as a mixture of diastereomers or a mixture enriched in diastereomers or a mixture enriched in enantiomers. A mixture enriched in enantiomers or a mixture of diastereomers may be separated where necessary, using methods know to the specialist in this field, so that the enantiomers or the diastereomers may be used separately. In those cases, where a carbon-carbon double bond is present, both the “cis” and the “trans” isomers are a part of this invention. In cases where tautomeric forms may exist, as for example in keto-enol tautomerism, all the tautomeric forms are included in this invention, and these forms may exist in equilibrium or preferentially as one form.

Implantable infusion pumps as used herein are intended to be applied into the subcutaneous tissue and fixed thereby a minimal invasive surgical procedure. They are designed for a long operating life of up to 20 years. Implantable infusion pumps consist of a compressible drug reservoir of 10, 15 up to 40 mL made of titan bellows surrounded by an inert gas. The infusion pumps need no battery changes because the energy is automatically replaced with every refill. The body temperature causes the gas to expand and putting pressure on the drug reservoir. The flow rates are regulated by a capillary chip for a continuous drug flow without fluctuations at rates of 0.25 mL up to 0.8 mL per 24 hours. The typical weight is about 85 g for a 10 mL reservoir pump. Other elements are there-filling port with silicone septum, a filter and a needle stop. Due to the inert titan material, the infusion pumps can be operated at physiologically acceptable pH-values between 3 and 8 and are operable even during a MRI intervention. The dosage is individually adapted to each patient. Implantable infusion pumps need only to be re-filled every 1 to 12 weeks.

Such pumps are described e.g. in US 20150343139A1, U.S. Pat. Nos. 6,626,867B1, 6,283,949B1, 5,769,823A, DE 19635056 A1, and others.

The following examples can be repeated with similar success by substituting the generically or specifically described reactants and/or operating conditions. It is believed than one skilled in the art can easily ascertain the essential characteristics of these methods and understands the Examples of the invention as exemplary. Thus, the below examples are not limiting the described manufacturing ways.

### EXAMPLE 1

### Variant 1

Production of a Sterile Lyophilisate with L-DOPA Glycerin Ester for Infusion after Reconstitution Before Use

275.0 g of L-DOPA Glycerin ester is dissolved for injection purposes with 4.0 g of citric acid monohydrate, and 10 g of sodium citrate dihydrate in 1000 mL of water for injection. The colorless solution, which has a pH of between 4.5 and 5, is then filtered by a membrane filter and then by a sterile filter (0.2 μm) under aseptic conditions and filled to 10 mL in each case in suitable vials. After sealing with a suitable plug, the solution is freeze-dried by an appropriate lyophilisation process, whereby in a vial, a dry cake is produced from the formulation components. Then, the vials are sealed. In this way, a batch is produced with 100 vials (theoretical yield) with a single dose of 2.75 g of L-DOPA Glycerin ester (corresponding to 2.00 g Levodopa). The lyophilisate thus obtained can be reconstituted with, e.g. 10 mL water for injection in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 2

214.1 g of L-DOPA Methyl Ester is dissolved for injection purposes with 4.0 g of citric acid monohydrate, and 10 g of sodium citrate dihydrate in 1000 mL of water for injection. The colorless solution is then filtered by a membrane filter and then by a sterile filter (0.2 μm) under aseptic conditions and filled to 10 mL in each case in suitable vials. After sealing with a suitable plug, the solution is freeze-dried by an appropriate lyophilisation process, whereby in a vial, a dry cake is produced from the formulation components. Then, the vials are sealed. In this way, a batch is produced with 100 vials (theoretical yield) with a single dose of 2.14 g of L-DOPA Methyl Ester (corresponding to 2.00 g Levodopa). The lyophilisate thus obtained can be reconstituted with, e.g. 10 mL N-Methylpyrrolidone in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 3

431.0 g of L-DOPA Cholin Ester is dissolved for injection purposes with 4.0 g of citric acid monohydrate, 10 g of sodium citrate dihydrate and 10 g Glutathione (Ascorbic acid, Cysteine, Na bisulfite) in 1000 mL of water. The clear and slightly yellow solution is then filtered by a membrane filter and subsequently by a sterile filter (0.2 μm) under aseptic conditions and filled to 10 g in each case in suitable vials. After sealing with a suitable plug, the solution is freeze-dried by an appropriate lyophilisation process, whereby in a vial, a dry cake is produced from the formulation components. Then, the vials are sealed. In this way, a batch is produced with 100 vials (theoretical yield) with a single dose of 4.31 g of L-DOPA Cholin Ester (corresponding to 3.00 g Levodopa). The lyophilisate thus obtained can be reconstituted with, e.g. 20 mL N-Methylpyrrolidone in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 4

431.0 g of L-DOPA Cholin Ester and 10 g Glutathione (Ascorbic acid, Cysteine, Na bisulfite) are dissolved in 1000 mL of water for injection. The clear and slightly yellow solution is then filtered by a membrane filter and subsequently by a sterile filter (0.2 μm) under aseptic conditions and filled to 10 mL in each case in suitable vials. After sealing with a suitable plug, the solution is freeze-dried by an appropriate lyophilisation process, whereby in a vial, a dry cake is produced from the formulation components. Then, the vials are sealed. In this way, a batch is produced with 100 vials (theoretical yield) with a single dose of 4.31 g of L-DOPA Cholin Ester. The lyophilisate thus obtained can be reconstituted with, e.g. 20 mL N-Methylpyrrolidone (tert. Butanol/Water) in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### EXAMPLE 2: PRODUCTION OF A STERILE SOLUTION READY TO USE INJECTABLE WITH L-DOPA GLYCERIN ESTER (L-DOPA ETHYL ESTERL-DOPA CHOLIN ESTER) FOR INFUSION

### Variant 1

550.0 g of L-DOPA Glycerin ester and 100.0 g of Benserazide are dissolved in 2000 mL of N-Methylpyrrolidone, medical grade. 20 g Glutathione (Cysteine, Ascorbic acid) are added to the solution. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41.7 μL/hour and the system delivers continuously 275 mg L-DOPA Glycerin ester/day (=corresponding to 200 mg Levodopa) over 10 days before a refill of the pump as described is necessary.

### Variant 2

457.2 g of L-DOPA Ethyl Ester, 2.0 g Rasagiline and 100.0 g of Benserazide (EthylCarbidopa), are dissolved in 2000 mL of N-Methylpyrrolidone (Ethanol, ter. Butanol/Water, medical grade). 20.0 g Glutathione (Cysteine, Ascorbic acid) are added to the solution. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (e.g. tricumed; IP1000V). Following activation of the pump the infusion rate is set to 41.7 μl/h and the system delivers continuously 228.6 mg L-DOPA Ethyl Ester/day (=corresponding to 200 mg Levodopa) in combination with 1 mg Rasagiline/day over 10 days before a refill of the pump as described is necessary.

### Variant 3

574.7 g of L-DOPA Cholin Ester, 5.0 g Opicapone and 100.0 g of Benserazide (EthylCarbidopa), are dissolved in 2000 mL of N-Methylpyrrolidone (Ethanol, ^{tert.-}Butanol/Water, medical grade). 20.0 g Glutathione (Cysteine, Ascorbic acid) are added to the solution. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (e.g. tricumed; IP1000V). Following activation of the pump the infusion rate is set to 41.7 μL/h and the system delivers continuously 287.4 mg L-DOPA Cholin Ester/day (=corresponding to 200 mg Levodopa) in combination with 2.5 mg Opicapone/day over 10 days before a refill of the pump as described is necessary.

### EXAMPLE 3

Production of a Sterile Lyophilisate with L-DOPA Glycerin Ester and Benserazide for Infusion after Reconstitution Before Use

275.0 g of L-DOPA Glycerin ester and 50 g Benserazide is dissolved for injection purposes with 4.0 g of citric acid monohydrate, and 10 g of sodium citrate dihydrate in 1000 mL of water for injection. The colorless solution is then filtered by a membrane filter and then by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.75 g of L-DOPA Glycerin ester and 0.5 g of Benserazide. The lyophilizate thus obtained can be reconstituted with, e.g. water for injection (2-5 mL) in the vial and produces a solution that is appropriate for use for injection, infusion or percutaneous infusion by a PEG for immediate use, whereby the composition of the solution with the selected adjuvants produces adequate stability under conditions of use of at least 72 h.

### EXAMPLE 4

Production of a Sterile Lyophilisate with L-DOPA Glycerin Ester and a Micell Forming Excipient to Protect L-Dopa from Crystallisation During Application for Infusion During Use

275.0 g of L-DOPA Glycerin ester and 25 g Solutol HS 15 is dissolved for injection purposes with 4.0 g of citric acid monohydrate, and 10 g of sodium citrate dihydrate in 1000 mL of water for injection. The colorless solution is then filtered by a membrane filter and then by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.75 g of L-DOPA Glycerin ester. The lyophilisate thus obtained can be reconstituted with, e.g. water for injection (2-5 mL) in the vial and produces a solution that is appropriate for use for injection, infusion or percutaneous infusion by a PEG for immediate use, whereby the composition of the solution with the selected adjuvants produces adequate stability under conditions of use of at least 72 h.

### EXAMPLE 5

Production of a Sterile Lyophilisate with L-DOPA Glycerin Ester and an Antioxidant (L-Cysteine, Glutathione, Ascorbic Acid) for Infusion after Reconstitution Before Use

275.0 g of L-DOPA Glycerin ester and 2 g L-Cysteine is dissolved for injection purposes with 4.0 g of citric acid monohydrate, and 10 g of sodium citrate dihydrate in 1000 mL of water for injection. The colorless solution is then filtered by a membrane filter and then by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.75 g of L-DOPA Glycerin ester. The freeze-dried product thus obtained can be reconstituted with, e.g. water for injection (2-5 mL) in the vial and produces a solution that is appropriate for use for injection, infusion or percutaneous application by a PEG for immediate application using e.g. a patch pump, whereby the composition of the solution with the selected adjuvants produces adequate stability under conditions of use of at least 72 h.

### EXAMPLE 6

### Enzymatic Cleavage of L-DOPA Glycerin Ester in Human Plasma (Ex Vivo) in Comparison to L-Dopa Ethyl Ester

2.0 mL Plasma-samples (n=3) were mixed with 40 μL L-DOPA Glycerin ester and incubated at pH 7.4 and 37° C. After 2, 15, 30, 45, 60 and 120 min 200 μL sample were taken, immediately deproteinized (200 μL 0.4N PCA), precipitated (200 μL 0.1% formic acid) and centrifuged. 10 μL of the supernatant was analyzed by UPLC-PDA-MS/MS for its content of L-DOPA Glycerin ester and L-Dopa. For purpose of comparison L-Dopa Ethyl Ester was investigated under same conditions.

L-DOPA Glycerin ester is converted to L-Dopa in human plasma to L-Dopa at body temperature practically at the same rate and extent compared to the known L-Dopa Ethyl Ester under the conditions chosen. After 2 hours 60 (50)% of L-DOPA Glycerin ester (L-Dopa Ethyl Ester) has been converted to L-Dopa. (FIG. 1)

### EXAMPLE 7

### Improving Local Tolerability

### Variant 1

### Application of L-DOPA Ester by a Twin-Chamber Infusion Pump

E.g., 5.5 g of L-DOPA Glycerin ester prepared according to Example 1 are reconstituted in 5 mL of water for injection and transferred to the first cartridge of a twin chamber pump (e.g. Cane Crono Twin pump). 5 mL of a sterile phosphoric buffer (at least 0.5 mol/I) having sufficient buffer capacity (to produce after mixing with the L-DOPA Glycerin ester an infusion solution with a pH around 7.4) is filled into the second cartridge. The twin chamber pump is operating in a way that the solution of cartridge 1 and cartridge 2 are mixed before they are applied by infusion. The twin chamber pump delivers both solutions with a flow rate between 50 and 500 μL/hour to the subcutaneous tissue.

### Variant 2

Application of L-DOPA Ester in an Infusion-Pump System which Allows Changing the Infusion Site Every 6 (12, 24 or More h) to Improve Local Tolerability.

E.g., 2.75 g of L-DOPA Glycerin ester prepared according to Example 1 are reconstituted in 5 mL of water for injection and transferred to a patch pump, which operates with an exchangeable reservoir attached to the injection needle system (e.g. Kaleido, ViCentra). The pump delivers the solution in a typical flow rate, however the reservoir with the needle injection system is exchanged every 6, 12, 24 h or more hours allowing different sites of the infusion to improve local tolerability.

### Variant 3

Continuous L-DOPA Application with Short Flow Intervals with Very Low Infusion Rated During the Infusion to Improve Local Tolerability.

E.g., 2.75 g of L-DOPA Glycerin ester prepared according to Example 1 are reconstituted in 5 mL of water for injection and transferred to a patch pump, which allows flow down regulation to minimal flow rates. Than short minimal infusion rate intervals from e.g. 1 min up to 15 (30/45) minutes duration are used to allow the tissue at the infusion site to recover and are followed by a higher infusion rate to reach an average flow rate of e.g. 50 and 500 μl/h. This procedure improves the local tolerability and does not hamper a smoothened L-DOPA plasma level

### EXAMPLE 8

### Application of L-DOPA Glycerin Ester Using PEG Application

Dissolve 13.6 mg of potassium dihydrogen phosphate in 2000.00 mg water. The resulting solution is used to dissolve 1.38 g of L-DOPA Glycerin ester.

The resulting solution is sterilized by sterile filtration.

The resulting solution can be applied by percutaneous endoscopic gastrostomy (PEG) using a portable mini-pump (e.g. patch pump) in combination with an appropriate PEG catheter or tube system. The solution may be supplied either in the stomach or into the upper intestine, this is typically done via a percutaneous endoscopic gastrostomy (PEG).

### EXAMPLE 9

### Application of L-DOPA Glycerin Ester Using JET-PEG Application

Dissolve 13.6 mg of potassium dihydrogen phosphate in 2000.00 mg water. The resulting solution is used to dissolve 2.75 g of L-DOPA Glycerin ester.

The resulting solution is sterilized by sterile filtration.

The resulting solution is applied to the upper part of the small intestine (JET-PEG) either via the stomach or the solution is given percutaneously by a specific PEJ-tube directly to the jejunum.

### EXAMPLE 10

### Production of L-DOPA Cholin Ester pH 7

Dissolve 10.40 mg of anhydrous disodium hydrogen phosphate 7.30 mg of sodium dihydrogen phosphate monohydrate 2.00 g of water and adjust the pH with phosphoric acid to pH 7.0. Dissolve 575.0 mg L-DOPA Cholin ester in the solution. The resulting solution is sterilized by sterile filtration and used as a 1-day application system, e.g. in a portable infusion pump.

### EXAMPLE 11

### Production of L-DOPA Cholin Ester pH 4.5

Dissolve 13.6 mg of potassium dihydrogen phosphate in 2.0 g water. The resulting solution adjusted to pH 4.5 is used to dissolve 1.15 g of L-DOPA Cholin ester.

The resulting solution is sterilized by sterile filtration and used as a 1-day application system, e.g. in a portable infusion pump.

### EXAMPLE 12

Production of L-DOPA Glycerin Ester Combined with Rasagiline (as Tartrate)

Dissolve 1.15 g of L-DOPA Glycerin ester in 6000.00 mg water. The resulting solution is used to dissolve 5.61 mg of Rasagiline tartrate.

The resulting solution is sterilized by sterile filtration and used as a 3-day application system, e.g. in a portable infusion pump.

### EXAMPLE 13: PRODUCTION OF A STERILE LYOPHILISATE WITH L-DOPA GLYCERIN ESTER FOR INFUSION AFTER RECONSTITUTION BEFORE USE

### Variant 1

275.0 g of L-DOPA Glycerin ester is dissolved for injection purposes with 4.0 g of citric acid monohydrate, and 10 g of sodium citrate dihydrate in 1000 mL of water for injection. The colorless solution, which has, is then filtered by a membrane filter and then by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.75 g of L-DOPA Glycerin ester (corresponding to 2.0 g of Levodopa). The lyophilisate thus obtained can be reconstituted with, e.g. water for injection in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 2

228.6 g of L-DOPA Ethyl Ester is dissolved for injection purposes with 4.0 g of citric acid monohydrate, and 10 g of sodium citrate dihydrate in 1000 mL of water for injection. The colorless solution is then filtered by a membrane filter and then by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.28 g of L-DOPA Ethyl Ester (corresponding to 2.0 g of Levodopa). The lyophilisate thus obtained can be reconstituted with, e.g. N-Methylpyrrolidone or ethanol in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 3

431.0 g of L-DOPA Cholin Ester HCl is dissolved for injection purposes 4.0 g of citric acid monohydrate, 10 g of sodium citrate dihydrate and 10 g Glutathione Ascorbic acid, Cysteine, Na bisulfite) in 1000 mL of water for injection. The clear and slightly yellow solution is then filtered by a membrane filter and subsequently by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 4.3 g of L-DOPA Cholin Ester. The lyophilisate thus obtained can be reconstituted with, e.g. N-Methylpyrrolidone or ethanol in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 4

275.0 g of L-DOPA Glycerin ester and 10 g Glutathione (Ascorbic acid, Cysteine, Na bisulfite) are dissolved in 1000 mL of water for injection. The clear and slightly yellow solution is then filtered by a membrane filter and subsequently by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.75 g of L-DOPA Glycerin Ester. The lyophilisate thus obtained can be reconstituted with, e.g. sterile N-Methylpyrrolidone (tert. Butanol/Water) in the vial and produces a solution that is appropriate for use for injection, continuous infusion or percutaneous infusion by a PEG for immediate use, whereby the composition of the solution with the selected adjuvants produces adequate stability under conditions of use of at least 72 hours.

### Variant 5

222.0 g of L-DOPA Glycerin ester, 10.0 g Apomorphine HCl, 0.5 g Levomefolat Na and 10.0 g Glutathione (Ascorbic acid, Cysteine, Na bisulfite) are dissolved in 1000 mL of water for injection. The clear and slightly yellow solution is then filtered by a membrane filter and subsequently by a sterile filter (0.2 μm) under aseptic conditions and filled to 10 mL in each case in suitable vials. After sealing with a suitable plug, the solution is frozen at minus 40-50° C. and then dried in a vacuum with use of a suitable freeze-dryer, whereby in a vial, a dry cake is produced from the formulation components. Then, the vials are sealed. In this way, a batch is produced with 100 vials (theoretical yield) with a single dose of 2.22 g of L-DOPA Glycerin ester. The lyophilisate thus obtained can be reconstituted with water for injection (sterile N-Methylpyrrolidone, tert. Butanol/Water) in the vial and produces a solution that is appropriate for use for injection, continuous infusion or percutaneous infusion by a PEG for immediate use, whereby the composition of the solution with the selected adjuvants produces adequate stability under conditions of use of at least 72 hours

### EXAMPLE 14: PRODUCTION OF A STERILE SOLUTION READY TO USE INJECTABLE WITH L-DOPA GLYCERIN ESTER (L-DOPA ETHYL ESTER, L-DOPA CHOLIN ESTER) FOR INFUSION (IMPLANTABLE PUMP)

### Variant 1

457.2 g of L-DOPA Ethyl Ester and 100.0 g of Benserazide are dissolved in 2000 mL of N-Methylpyrrolidone, medical grade. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41.7 μl/h and the system delivers continuously 229 mg L-DOPA Ethyl Ester/day (=corresponding to 200 mg Levodopa) over 10 days before a refill of the pump as described is necessary.

### Variant 2

457.2 g of L-DOPA Ethyl Ester and 100.0 g of EthylCarbidopa are dissolved in 2000 mL of N-Methylpyrrolidone, medical grade. The slightly yellow clear solution is then filtered filter and then by a sterile filter (0.2 μm) under aseptic conditions and then filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41.7 μl/h and the system delivers continuously 229 mg L-DOPA Ethyl Ester/day (=corresponding to 200 mg Levodopa) over 10 days before a refill of the pump as described is necessary.

### Variant 3

550 g of L-DOPA Ethyl Ester HCl, 2 g Rasagiline and 100 g of Benserazide (EthylCarbidopa), are dissolved in 2000 mL of N-Methylpyrrolidone (Ethanol, tert. Butanol/Water, medical grade. 20 g Glutathione (Cysteine, Ascorbic acid) is added to the solution. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (e.g. tricumed; IP1000V). Following activation of the pump the infusion rate is set to 41 μL/h and the system delivers continuously 275 mg L-DOPA Ethyl Ester/day (=corresponding to 200 mg Levodopa) in combination with 1 mg Rasagiline/day over 10 days before a refill of the pump as described is necessary.

### Variant 4

626 g of L-DOPA Cholin Ester HCl, 5 g Opicapone and 100 g of Benserazide (EthylCarbidopa), are dissolved in 2000 mL of N-Methylpyrrolidone (Ethanol, ter. Butanol/Water, medical grade. 20 g Glutathione (Cysteine, Ascorbic acid) is added to the solution. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (e.g. tricumed; IP1000V). Following activation of the pump the infusion rate is set to 41 μL/h and the system delivers continuously 313 mg L-DOPA Cholin Ester/day (=corresponding to 200 mg Levodopa) in combination with 2.5 mg Opicapone/day over 10 days before a refill of the pump as described is necessary.

### EXAMPLE 15: PRODUCTION OF A STERILE SOLUTION READY TO USE INJECTABLE WITH L-DOPA ETHYL ESTER FOR INFUSION BY AN IMPLANTABLE MINIPUMP

### Variant 1

457.2 g of L-DOPA Ethyl Ester and 100 g of Benserazide are dissolved in 1000 mL of Ethanol. The slightly yellow clear solution is then filtered by a membrane filter and then by a sterile filter (0.2 μm) under aseptic conditions. The solution is filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41 μL/h and the system delivers continuously 457 mg L-DOPA Ethyl Ester/day (=corresponding to 400 mg Levodopa) over 10 days before a refill of the pump as described is necessary.

### Variant 2

457.2 g of L-DOPA Ethyl Ester and 100 g of EthylCarbidopa are dissolved in 1000 mL of Ethanol. The slightly yellow clear solution is then filtered filter and then by a sterile filter (0.2 μm) under aseptic conditions and then filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41 μL/h and the system delivers continuously 457 mg L-DOPA Ethyl Ester/day (=corresponding to 400 mg Levodopa) over 10 days before a refill of the pump as described is necessary.

### Variant 3

457.2 g of L-DOPA Ethyl Ester and 100 g of EthylCarbidopa are dissolved in 1000 mL of Ethanol. The slightly yellow clear solution is then filtered filter and then by a sterile filter (0.2 μm) under aseptic conditions and then filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41 μl/h and the system delivers continuously 457 mg L-DOPA Ethyl Ester/day (=corresponding to 400 mg Levodopa) over 10 days before a refill of the pump as described is necessary.

### Variant 4

229 g of EthylDopa, 25 g of EthylCarbidopa and 1 g of potassium-glycerolat (guanidine-carbonat, kreatin-citrate) as a catalysator are dissolved in 1000 ml of water-free Glycerol medical grade. The slightly yellow clear solution is then filtered by a membrane filter under aseptic conditions and then filled to 10 ml into injection vials and then terminal sterilized at 121° C. for 20 minutes (or 80° C. for 60 minutes) in an autoclave giving a ready to use sterile formulation of L-Dopa Glycerin Ester (GlyDopa) and Carbidopa-glycerolester (with a minor content of EthylDopa and EthylCarbidopa). The vials are stored protected from light at controlled temperatures between 2-25° C. (most preferable between 2-8° C.). 10 ml of the sterile solution is filled through the filling septum into the reservoir of an implantable infusion pump (tricumed; IP1000V). Following activation of the pump the infusion rate is set to 41 μl/h and the system delivers continuously 275 mg GlyDopa/day (=corresponding to 200 mg LevoDopa) over 10 days before a refill of the pump as described is necessary.

### Variant 5

229 g of EthylDopa, 25 g of EthylCarbidopa and 1 g of potassium-glycerolat (guanidine-carbonat, kreatin-citrate) as a catalysator are dissolved in 1000 ml of water-free Glycerol medical grade. The slightly yellow clear solution is then filtered by a membrane filter under aseptic conditions and then filled to 10 ml into injection vials and then terminal sterilized at 121° C. for 20 minutes (or 80° C. for 60 minutes) in an autoclave giving a ready to use sterile formulation of GlyDopa and Carbidopa-glycerolester (with a minor content of EthylDopa and EthylCarbidopa). The vials are stored protected from light at controlled temperatures between 2-25° C. (most preferrable between 2-8° C.). 10 ml of the sterile solution is filled into the reservoir of an electronic minipump (Cane Crono 5®). Following activation of the pump the infusion rate is set to 138 μl/h and the system delivers continuously 825 mg GlyDopa/day (=corresponding to 600 mg LevoDopa) over 72 hour before a refill of the pump is necessary

### Variant 6

457.2 g of L-DOPA Ethyl Ester and 100 g of Benserazide is dissolved in 1000 mL of Ethanol, medical grade. The slightly yellow clear solution is then filtered filter and then by a sterile filter (0.2 μm) under aseptic conditions and then filled to 10 mL into injection vials. The vials are stored protected from light at controlled temperatures between 2-25° C. (most preferable between 2-8° C.). 10 mL of the sterile solution is filled through the filling septum into the reservoir of an implantable infusion pump (tricumed; IP1000V). Following activation of the pump the infusion rate is set to 41 μl/h and the system delivers continuously 457 mg L-DOPA Ethyl Ester/day (=corresponding to 400 mg Levodopa) over 10 days before a refill of the pump as described is necessary.

### EXAMPLE 16: STABILITY OF A STERILE READY TO USE INJECTABLE WITH L-DOPA ETHYL ESTER DURING THE APPLICATION PERIOD

### Variant 1

An injection vial containing a sterile solution of 660 mg L-DOPA Ethyl Ester in 1 mL Ethanol was stored protected from light at a controlled room temperature (25° C.) for 7 days. Thereafter the concentration of L-DOPA Ethyl Ester in the sterile solution was determined by using a specific HPLC method. The purity of the active remained at a level between 95 to 99% of the initial value.

### Variant 2

An injection vial containing a sterile solution of 330 mg L-DOPA Ethyl Ester in 1 mL N-Methlypyrrolidone was stored protected from light at a controlled room temperature (25° C.) for 7 days. Thereafter the concentration of L-DOPA Ethyl Ester in the sterile solution was determined by using a specific HPLC method. The purity of the active remained at a level between 94 to 98% of the initial value.

1.-13. (canceled)

14. A method of treating a neurological disease in a mammal, comprising continuously administering to said mammal an amount of a compound of general Formula I 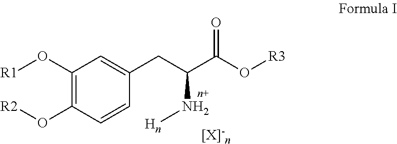
wherein [X]⁻ is a physiologically compatible anion,
wherein n is 0 or 1,
wherein R1 and R2 are independently of each other, selected from the group comprising hydrogen, or hydrogensulfate, phosphate, hydrogen phosphate, dihydrogen phosphate benzoate, formate, acetate, propionate, butanoate, valerate, silyl,
or
R1, R2 together are hydrogen phosphate, sulfate, methylene, isopropylidene, wherein R3 represents a methyl-, ethyl-, n-propyl-, i-propyl-, n-butyl, i-butyl or t-butyl group or an unbranched, branched or cyclic polyhydroxyl residue with 1-12 carbon atoms and 1-6 OH-groups which can further be substituted by unsaturated groups, halogens or organic functional groups like carboxylic group and aldehyde.

15. The method according to claim 14 in which the physiologically compatible anion [X]⁻ is selected from the group consisting of halogenide, sulfate, hydrogensulfate, phosphate, hydrogen phosphate, dihydrogen phosphate, carboxylate like benzoate, formate, acetate, propionate, butanoate, valerate, myristate, octoate, stearate, ascorbate, trifluoracetate, phosphonate, phosphoric acid ester, sulfonate (e.g. tosylate) or sulfuric acid ester (e.g. ethyl sulfate).

16. The method according to claim 14, wherein R3 represents an unbranched, branched or cyclic polyhydroxyl residue comprising Glyceryl, C₄-alkyl carrying 3-4 OH-groups, C₆-alkyl carrying 3-6 OH-groups, monosaccharidyl, disaccharidyl and oligosaccharidyl (cyclic, open-chained) as well as derivatives of polyhydroxyl compounds such as acetonides (e.g. solketal residue), methylal (e.g. glycerin methylal residue), carbonates (e.g. glycerin carbonate residue) as well as orthoester and ethyliden acetale of vicinal OH-groups, wherein the polyhydroxy compounds may be further substituted by keto, ketal, amino, thio, sulfate and phosphate residues, which can further be substituted by unsaturated groups, halogens or organic as well as inorganic functional groups like carboxylic group, phosphate, phosphonate, sulfate, sulfonate and derivatives thereof and wherein one hydroxyl residue of R3 can be replaced by an ammonium cation.

17. The method according to claim 14, wherein R3 is selected from the group comprising glyceryl, erithryl, trihydroxymethyl methyl, pentaerithryl, glucosyl, fructosyl, glycerin methylal, choline, glycerine phosphate, glycerine sulfate, 2,3-dihydroxypropyl 2′-trimethylazaniumylethyl phosphate and solketyl.

18. The method according to claim 14,
wherein [X]⁻ is Cl⁻, R1 and R2 are both hydrogen and R3 is glyceryl, or,
wherein [X]⁻ is Cl⁻, R1 and R2 are Hydrogen and R3 is cholenyl chloride residue, or
wherein [X]⁻ is Cl⁻, R1 and R2 are Hydrogen and R3 is 2,3-dihydroxypropyl 2′-trimethylazaniumylethyl phosphate residue.

19. The method according to claim 14, wherein the solvent for parenteral or percutaneous endoscopic gastrostomy (PEG) application is, an aqueous buffer solution (pH 2-5) or an aprotic biocompatible organic solvent or a mixture of any of these suitable solvents.

20. The method according to claim 14, wherein continuous administration is done by injection or infusion which is achieved by portable pumps, implanted pumps or patch pumps.

21. The method according to claim 14, wherein continuous administration is done by injection or infusion which is achieved by portable pumps, implanted pumps or patch pumps enabling continuous delivery by a constant flowrate or patient individual time-adjustable day-profile.

22. The method according to claim 14, wherein a compound of formula I is administered in combination with other oral or parenteral PD therapies.

23. The method according to claim 22, wherein said combination is further combined with folic acid and its derivatives, ascorbic acid or other antioxidants and stabilizers.

24. The method according to claim 14, wherein the neurological diseases is selected from Restless Legs Syndrome (RLS) or of related motor disorders or of Prolactinomas or in the treatment or prevention of OFF phenomena and especially akinetic crisis in PD patients.

25. The method according to claim 14, wherein the continuous application mode is selected from PEG (Percutaneous Endoscopic Gastrostomy) or injection or infusion.

26. The method according to claim 14, wherein the continuous application is achieved by an implantable mini-pump or patch pump.

27. A method of treating a neurological disease in a mammal, comprising continuously administering to said mammal an amount of a compound of general Formula I 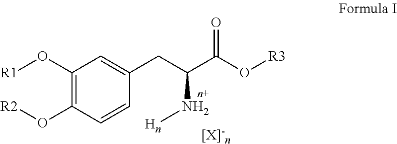
wherein [X]⁻ is a physiologically compatible anion,
wherein n is 0 or 1,
wherein R1 and R2 are independently of each other, selected from the group comprising hydrogen, or hydrogensulfate, phosphate, hydrogen phosphate, dihydrogen phosphate benzoate, formate, acetate, propionate, butanoate, valerate, silyl,
or
R1, R2 together are hydrogen phosphate, sulfate, methylene, isopropylidene, wherein R3 represents a methyl-, ethyl-, n-propyl-, i-propyl-, n-butyl, i-butyl or t-butyl group or an unbranched, branched or cyclic polyhydroxyl residue with 1-12 carbon atoms and 1-6 OH-groups which can further be substituted by unsaturated groups, halogens or organic functional groups like carboxylic group and aldehyde
in combination with
MAO-B inhibitors
COMT-Inhibitors
or
Decarboxylase Inhibitors.

28. The method according to claim 27 in which the physiologically compatible anion [X]⁻ is selected from the group consisting of halogenide, sulfate, hydrogensulfate, phosphate, hydrogen phosphate, dihydrogen phosphate, carboxylate like benzoate, formate, acetate, propionate, butanoate, valerate, myristate, octoate, stearate, ascorbate, trifluoracetate, phosphonate, phosphoric acid ester, sulfonate (e.g. tosylate) or sulfuric acid ester (e.g. ethyl sulfate).

29. The method according to claim 27, wherein R3 represents an unbranched, branched or cyclic polyhydroxyl residue comprising Glyceryl, C₄-alkyl carrying 3-4 OH-groups, C₆-alkyl carrying 3-6 OH-groups, monosaccharidyl, disaccharidyl and oligosaccharidyl (cyclic, open-chained) as well as derivatives of polyhydroxyl compounds such as acetonides (e.g. solketal residue), methylal (e.g. glycerin methylal residue), carbonates (e.g. glycerin carbonate residue) as well as orthoester and ethyliden acetale of vicinal OH-groups, wherein the polyhydroxy compounds may be further substituted by keto, ketal, amino, thio, sulfate and phosphate residues, which can further be substituted by unsaturated groups, halogens or organic as well as inorganic functional groups like carboxylic group, phosphate, phosphonate, sulfate, sulfonate and derivatives thereof and wherein one hydroxyl residue of R3 can be replaced by an ammonium cation.

30. The method according to claim 27, wherein R3 is selected from the group comprising glyceryl, erithryl, trihydroxymethyl methyl, pentaerithryl, glucosyl, fructosyl, glycerin methylal, choline, glycerine phosphate, glycerine sulfate, 2,3-dihydroxypropyl 2′-trimethylazaniumylethyl phosphate and solketyl.

31. The method according to claim 27,
wherein [X]⁻ is Cl⁻, R1 and R2 are both hydrogen and R3 is glyceryl, or,
wherein [X]⁻ is Cl⁻, R1 and R2 are Hydrogen and R3 is cholenyl chloride residue, or
wherein [X]⁻ is Cl⁻, R1 and R2 are Hydrogen and R3 is 2,3-dihydroxypropyl 2′-trimethylazaniumylethyl phosphate residue.

32. The method according to claim 27,
wherein the MAO-B inhibitor, COMT-Inhibitor, or Decarboxylase Inhibitor is selected from Benserazide, Carbidopa, Carbidopaethylester, Methyldopa, α-Difluoromethyl-DOPA, Rasagiline, Selegiline, entacapone, colcapone, opicapone, tolcapone, apomorphine, nitecapone.

33. The method according to claim 27, wherein the solvent for parenteral or percutaneous endoscopic gastrostomy (PEG) application is, an aqueous buffer solution (pH 2-5) or an aprotic biocompatible organic solvent or a mixture of any of these suitable solvents.

34. The method according to claim 27, wherein continuous administration is done by injection or infusion which is achieved by portable pumps, implanted pumps or patch pumps.

35. The method according to claim 27, wherein continuous administration is done by injection or infusion which is achieved by portable pumps, implanted pumps or patch pumps enabling continuous delivery by a constant flowrate or patient individual time-adjustable day-profile.

36. The method according to claim 27, wherein the combination of a compound of formula I with MAO-B inhibitors, COMT-Inhibitors, or Decarboxylase Inhibitors is administered in combination with other oral or parenteral PD therapies.

37. The method according to claim 36, wherein said combination is further combined with folic acid and its derivatives, ascorbic acid or other antioxidants and stabilizers.

38. The method according to claim 27, wherein the neurological diseases is selected from Restless Legs Syndrome (RLS) or of related motor disorders or of Prolactinomas or in the treatment or prevention of OFF phenomena and especially akinetic crisis in PD patients.

39. The method according to claim 27, wherein the continuous application mode is selected from PEG (Percutaneous Endoscopic Gastrostomy) or injection or infusion.

40. The method according to claim 27, wherein the continuous application is achieved by an implantable mini-pump or patch pump.
